(19) United States
(12) Patent Application Publication
(10) Pub. No.: US 2023/0314422 A1
(43) Pub. Date: Oct. 5, 2023

(54) METHODS FOR RAPID ANALYTE CONCENTRATION ANALYSIS FOR MULTIPLE SAMPLES

(71) Applicant: Biosensing Instrument Inc., Tempe, AZ (US)

(72) Inventors: Nguyen Ly, Tempe, AZ (US); Tianwei Jing, Tempe, AZ (US); Feimeng Zhou, Temple City, CA (US)

(73) Assignee: Biosensing Instrument Inc., Tempe, AZ (US)

(21) Appl. No.: 18/130,396

(22) Filed: Apr. 3, 2023

Related U.S. Application Data
(60) Provisional application No. 63/326,909, filed on Apr. 3, 2022.

(51) Int. Cl. G01N 33/543; B01L 3/00

(52) U.S. Cl. CPC G01N 33/54366; B01L 3/502715; B01L 3/502761; B01L 3/50273; B01L 2300/0816; B01L 2400/0487; B01L 2300/0861; B01L 2400/06; B01L 2300/0627

(57) ABSTRACT

A method can include measuring increments in a response signal in multiple sample injection sessions in a sensing channel until the response signal reaches a threshold response capacity. Measuring the increments can include: (a) starting a respective sample injection session of the multiple sample injection sessions by injecting a sample with an analyte to the sensing channel; (b) controlling the valve port to terminate the respective sample injection session; (c) measuring the response signal based on a reaction between the sample and the ligand; and/or (d) upon determining that the response signal is not greater than the threshold response capacity, determining a respective response increment of the increments for the respective sample injection session, and starting a subsequent session of the multiple sample injection sessions for determining a subsequent increment of the increments. The method further can include determining an analyte concentration of the sample based at least in part on the increments. Other embodiments are disclosed.

### CROSS-REFERENCE TO RELATED APPLICATIONS

This application claims priority to U.S. Provisional Patent Application No. 63/326,909, filed Apr. 3, 2022. U.S. Provisional Patent Application No. 63/326,909 is incorporated herein by reference in its entirety.

### TECHNICAL FIELD

This disclosure relates generally to methods using a multi-channel microfluidic system for determining analyte concentrations in multiple samples.

### BACKGROUND

Many existing techniques for determining analyte concentrations rely on labels (e.g., a chromophore, or a fluorescent, electroactive or radioactive molecule, etc.) pre-attached to the analytes (e.g., antibodies, such as enzyme-linked antibodies, total immunoglobin G (IgG) antibodies, and/or severe acute respiratory syndrome coronavirus 2 (SARS-CoV-2) antibodies). Examples of label-based techniques can include enzyme-linked immunosorbent assay (ELISA), and chemiluminescence immunoassay (CLIA). Label-free techniques also exist and generally involve measuring one or more universal physical properties (e.g., the mass, dielectric constant, refractive index, and/or viscoelasticity) of an analyte. For example, surface plasmon resonance (SPR) analysis, a label-free technique, generally can include measuring changes in refractive index associated with the attachment of an analyte to a sensor surface. The SPR analysis further can include utilizing a pre-immobilized ligand (e.g., antibodies or other molecules) to capture with high specificity an analyte (e.g., an antigen or nucleic acid or a small organic compound) in a solution delivered by a flow system. By measuring changes in an SPR angle or a reflected light intensity, a plot (e.g., a sensorgram) of response signal versus time can be generated and analyzed to obtain relevant information on binding interactions (e.g., an affinity constant (K_{D}) and association and dissociation rate constants (kₐ and k_{d})) between the ligand and the analyte.

For label-based techniques, degradations of enzymatic activities and/or fluorescent intensities compromise the analytical performances, including relatively high cost in manufacturing and storing the test kits. Additionally, additional limitations for these methods can include the long time required for the incubation/washing step between cycles. Label-free methods are not better in terms of the time consumed.

In addition to the slow association process and excessive consumption of the sample, repeated regenerations of the sensor surface can prolong the time for the measurements and in some cases, render the sensor unusable eventually. This aspect of SPR measurements can significantly lower the sample throughput and increase the assay costs, especially for the applications when numerous samples need to be analyzed rapidly. However, these issues have not been sufficiently addressed in the past. Despite the recent development of highly advanced fluidic systems and a variety of sensor types, as well as numerous publications on various applications of SPR to a wide range of biomolecules, SPR and related surface analyses remain time-consuming and laborious and require good knowledge of surface chemistry and mass transfer limitations. Further, although existing SPR protocols, once optimized, can be acceptable for kinetic studies (e.g., to derive kₐ and k_{d}), they can be inadequate for determining concentrations of numerous samples (e.g., clinical samples), mass testing of infectious diseases, and/or screening of drug candidates because of the slow and laborious process. Therefore, a need exists for a system and a method configured to overcome the above-mentioned disadvantages of the existing surface analysis approaches.

### BRIEF DESCRIPTION OF THE DRAWINGS

To facilitate further description of the embodiments, the following drawings are provided in which:

FIG. 1 illustrates a plot of response versus time, according to an embodiment;

FIG. 2 illustrates a block diagram of a multi-channel microfluidic system, according to an embodiment;

FIG. 3 illustrates plots of response versus time for various ligand densities, according to an embodiment;

FIG. 4 illustrates plots of response versus time for various analyte concentrations, according to an embodiment;

FIG. 5 illustrates plots of response versus analyte concentration, according to an embodiment;

FIG. 6 illustrates a plot of response versus time for response signals measured in an association phase at a sensing channel, according to an embodiment;

FIG. 7 illustrates a plot of response versus time for response signals measured in an association phase at a sensing channel, according to an embodiment; and

FIG. 8 illustrates a flow chart for a method, according to an embodiment.

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

The terms “couple,” “coupled,” “couples,” “coupling,” and the like should be broadly understood and refer to connecting two or more elements mechanically and/or otherwise. Two or more electrical elements may be electrically coupled together, but not be mechanically or otherwise coupled together. Coupling may be for any length of time, e.g., permanent or semi-permanent or only for an instant. “Electrical coupling” and the like should be broadly understood and include electrical coupling of all types. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real-time” encompasses operations that occur in “near” real-time or somewhat delayed from a triggering event. In a number of embodiments, “real-time” can mean real-time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, five seconds, ten seconds, thirty seconds, one minute, two minutes, or five minutes.

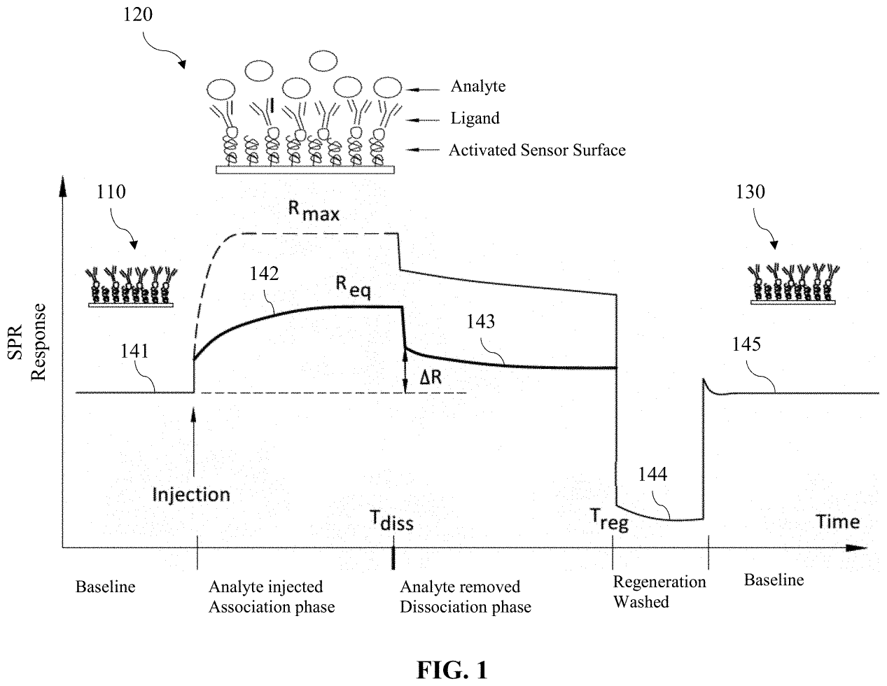

A typical protocol widely used in SPR analysis can include four steps. As shown in FIG. 1, the respective response corresponding to the four steps in the course of an exemplary reversible interaction at a sensing surface can be illustrated by a temporal plot, called an SPR sensorgram. The Y-axis of the SPR sensorgram indicates the response (e.g., the measured signal due to SPR angle changes or changes in the reflected light intensity), and the X-axis indicates the time. The first step for an exemplary SPR analysis can include: before the analyte is introduced onto a sensor surface, (a) pre-immobilizing a ligand onto the sensor surface (e.g., the sensor surface (110) in FIG. 1), and (b) maintaining a constant flow of analyte-free running buffer to stabilize a baseline (e.g., the response signal (141) in FIG. 1).

The second step for the exemplary SPR analysis can include: (a) delivering an analyte in a sample into a channel, and (b) ensuring the analyte in contact with the pre-immobilized ligand on the sensor surface (e.g., the sensor surface (120) in FIG. 1) so that binding can occur. This step is referred to as an association phase. In the association phase, binding events can be detected when the SPR response signal (e.g., the response signal (142) in FIG. 1) increases as a function of time. If sufficient time is given, the equilibrium phase can be established, and the SPR response signal can reach to an equilibrium binding signal (R_{eq}). If the analyte concentration is sufficiently high (e.g., 1 few μM for a reaction with the nM-level binding affinity or 1 μg/mL to 16 μg/mL of an antibody), all of the available binding sites on the sensor can be occupied by the analyte, and the signal can reach a maximum binding signal (Rₘₐₓ). Generally the association phase can take many minutes and even hours before the interaction reaches equilibrium, and until then, a constant delivery of the sample must be maintained. As a result, the exemplary SPR analysis can take a long time, and the quantity of the sample used can be large. Further, if the analyte concentration is too low (e.g., less than 0.1 nM for a binding reaction with nM affinity), it can become impractical, if not impossible, for the interaction to reach equilibrium (R_{eq}) within a reasonable measurement timeframe (e.g., <1 day).

Further, the third step for the exemplary SPR analysis can include: after the sample delivery is complete, flushing the sensor surface immediately with the analyte-free running buffer to allow only dissociation of the analyte from the ligand. This step is referred to as a dissociation phase. The dissociation can cause the response signal (e.g., the response signal (143) in FIG. 1) to decay as a function of time.

The fourth step for the exemplary SPR analysis can include: “washing” the sensor surface with a reagent solution for “regeneration” so that the original surface becomes again free of any analyte (e.g., the response signal (144) in FIG. 1). In so doing, the sensor surface can be recovered back to its original state (e.g., the sensor surface (130) and the response signal (145) in FIG. 1) and can be reused for subsequent measurements.

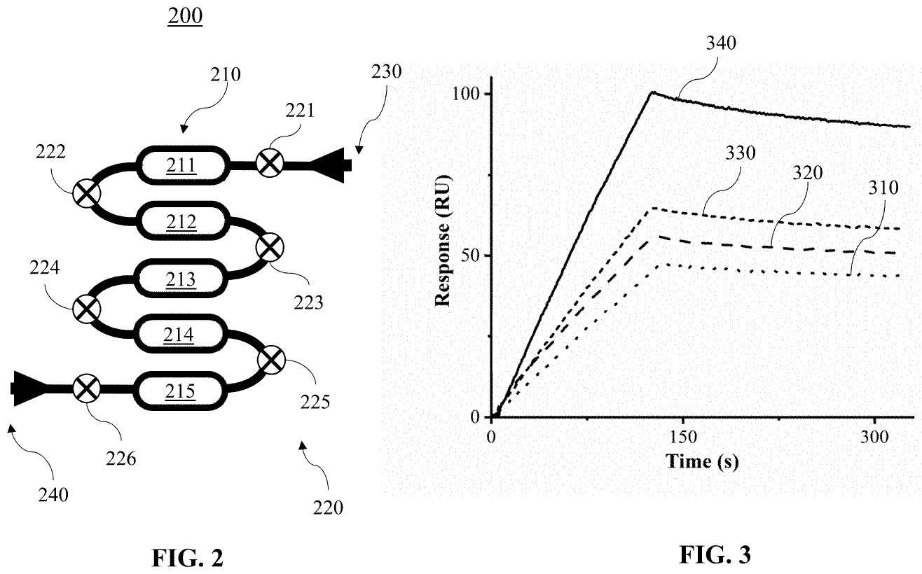

Various embodiments can include a method for facilitating analysis of multiple samples via a sensor with multiple sensing channels (e.g., a 5-channel microfluidic system in FIG. 2) without surface regenerations. In particular, unlike conventional methods that determine analyte concentration at an equilibrium phase, the method in the various embodiments can determine analyte concentration based on SPR measurements at multiple short cycles. That is, for example, the method can include repeatedly exposing a respective sample to each sensing channel of the sensor for a short period of time (e.g., less than 20 seconds, 30 seconds, 45 seconds, or 60 seconds, etc.) to measure the respective response at an early stage (e.g., 0-20 seconds, 0-25 seconds, or 0-60 seconds, etc.) or within a short period of time of an association phase. The method also can include, as another example, not regenerating the sensor surface until the overall binding response signal in each sensing channel reaches a predetermined threshold. Without the slow process of reaching equilibrium and with less frequent regeneration, the method can improve the surface techniques by rapid analyses of multiple samples and reduced sample consumption.

The analyte concentration can be determined quickly here because the analyte concentration is generally linearly proportional to the SPR response signal value at an early stage of an association phase. In binding kinetic theory, the interactions (association and/or dissociation) between an immobilized ligand (B) and an analyte (A) can be expressed as Equation (1) below:

$$A + B \begin{matrix}{⟶}^{{k}_{a}} \\ {⟵}_{{k}_{d}}\end{matrix} AB , \qquad ( 1 )$$

where kₐ is an association rate constant; and k_{d} is a dissociation rate constant.

During the association phase, the amount of a complex (AB) can scale with an SPR response signal (R) and be given by Equation (2) below:

$$R = \frac{{k}_{a} C {R}_{max}}{{k}_{a} C + {k}_{d}} ( 1 - {e}^{- ( {k}_{a} C + {k}_{d} ) t} ) , \qquad ( 2 )$$

where C is an analyte concentration; Rₘₐₓ is the maximum response signal value, associated with the maximum amount of the AB complex form on a sensor surface; and t is the reaction time.

At the early stage or within a short period of time (t) of the association phase, Equation (2) above can be simplified to Equation (3) below:

R=RₘₐₓkₐCt (3)

As such, because Rₘₐₓ, kₐ, and t can be constants and/or predetermined, the analyte concertation (C) can be determined after the SPR response signal (R) is measured at the early stage or within the short measurement cycle (t).

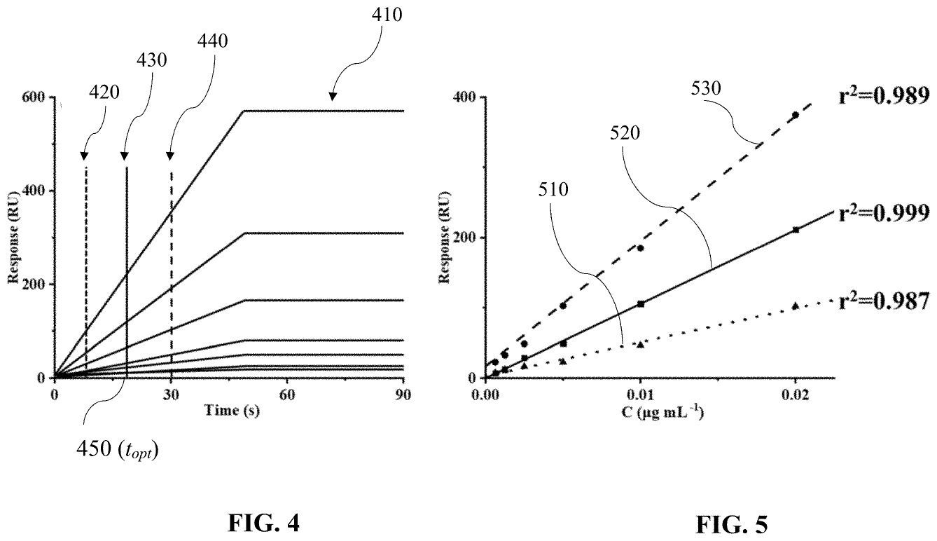

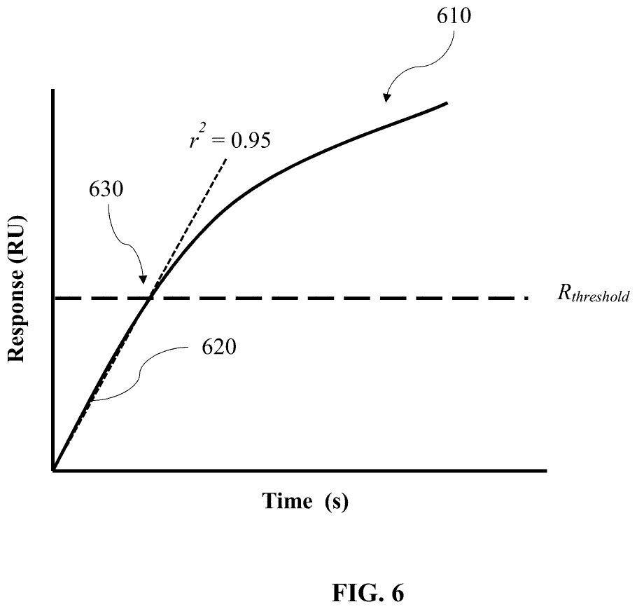

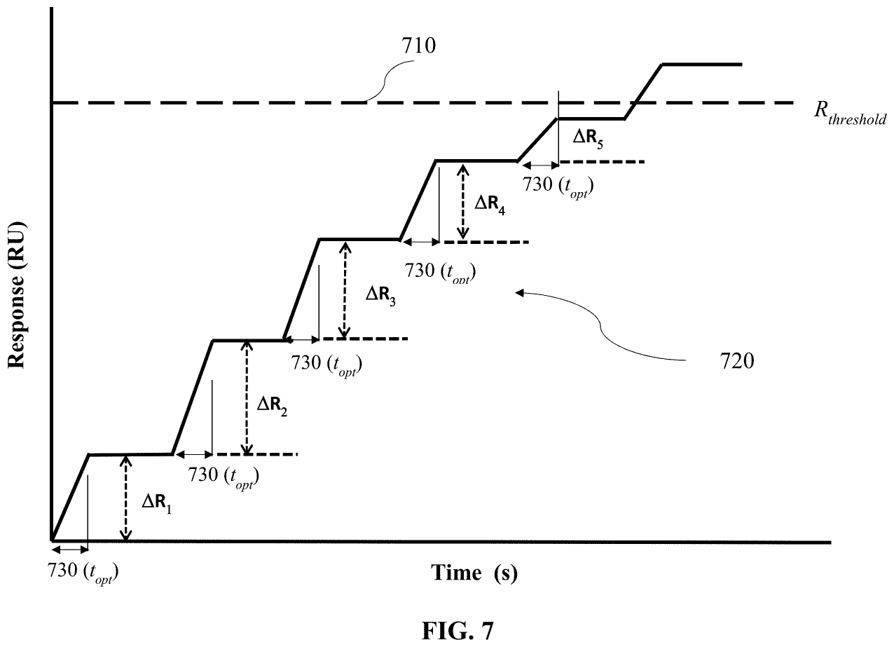

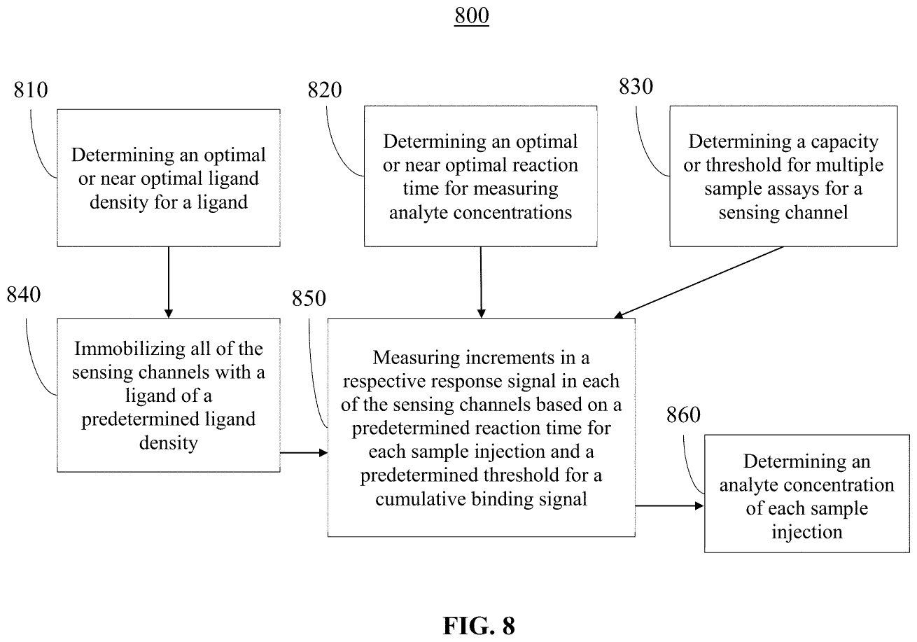

In many embodiments, a method (e.g., method 800 in FIG. 8) for facilitating high-throughput analysis of samples via a sensor with multiple sensing channels can include measuring analyte concentrations by: (a) immobilizing all of the sensing channels with a ligand of a predetermined ligand density (e.g., block 840 in FIG. 8); (b) measuring increments in a respective response signal in each of the sensing channels based on a predetermined reaction time (e.g., tₒₚₜ (450) in FIG. 4 or tₒₚₜ(730) in FIG. 7) for each sample injection and a predetermined threshold for a cumulative binding signal (e.g., R_{threshold} (630) in FIG. 6 or R_{threshold} (710) in FIG. 7) (e.g., block 850 in FIG. 8); and (c) determining an analyte concentration of each sample injection (e.g., block 860 in FIG. 8). In some embodiments, the method further can include preparing, before detecting the analyte concentrations, by one or more of: (a) determining an optimal or near optimal ligand density for a ligand (e.g., the above-mentioned predetermined ligand density) (e.g., block 810 in FIG. 8); (b) determining an optimal or near optimal reaction time for measuring analyte concentrations (e.g., the above-mentioned predetermined reaction time) (e.g., block 820 in FIG. 8); and/or (c) determining a capacity or threshold for multiple sample assays for a sensing channel (e.g., the above-mentioned predetermined threshold) (e.g., block 830 in FIG. 8).

In a number of embodiments, determining the optimal, or near optimal, ligand density (e.g., block 810 in FIG. 8) can include: (a) immobilizing a ligand (e.g., an antibody, a molecule, a protein, or histidine (His)-tagged SARS-CoV-2 spike (Si) protein) with a respective density of multiple candidate ligand densities (e.g., 0.500, 0.725, 0.900, 1.000, or 1.250 ng/mm²) onto a respective sensor surface of each of multiple sensing channels; (b) injecting an analyte solution (e.g., a solution with an antigen, nucleic acid, or a small organic compound, etc.) of a predetermined concentration (e.g., 1, 8, 12, 16, or 20 μg/mL) to the sensing channels; and (c) determining the optimal, or near optimal, ligand density based on a respective binding response signal on each sensing channel.

In several embodiments, the sensor surface of each of the multiple sensing channels (e.g., sensing channels (210) in FIG. 2) can include any suitable sensor surface treatment(s) for SPR analysis. An exemplary sensor surface can be covered with a tris-nitrilotriacetic acid (tris-NTA), single NTA, or thiol-terminated thin film, and another exemplary sensor surface can be coated with carboxymethylated dextran or mixed monolayers of polyethylene glycols, etc. In certain embodiments, a multi-channel microfluidic system (e.g., a five-channel microfluidic system (200) in FIG. 2) can be used to implement one or more of the acts in the method. The sensing channels (e.g., the sensing channels (210, 211, 212, 213, 214, and 215) of the system (200) in FIG. 2) of the multi-channel microfluidic system can be connected serially to allow a buffer to flow over the sensing channels. The multi-channel microfluidic system further can include two inlets (e.g., the inlets (230 and 240) in FIG. 2) to each receive a buffer flow over the sensing channels. Each of the inlets further can be connected to an auto-sampler to enable analyte delivery into the sensing channels and/or configured to control a speed of the buffer flow. The multi-channel microfluidic system additionally can include multiple valve ports (e.g., the valve ports (220, 221, 222, 223, 224, 225, and/or 226) in FIG. 2) respectively located on each end of the sensing channels (e.g., the sensing channels (210, 211, 212, 213, 214, and/or 215) of the system (200) in FIG. 2). The multi-channel microfluidic system further can be configured so that during the measurements of response signals, only one of the valve ports (e.g., the valve ports (220, 221, 222, 223, 224, 225, and/or 226) in FIG. 2) is open and the rest remain(s) closed.

In some embodiments, to immobilize the ligand for determining the optimal, or near optimal, ligand density, the method further can include immobilizing the ligand on the sensor surfaces of a plurality of sensing channels with different ligand densities. The plurality of sensing channels can include some or all of the multiple sensing channels of the sensor. With the plurality of sensing channels used, and each sensor surface including a different ligand density, the respective binding response signal for each ligand density can be measured simultaneously, and the optimal, or near optimal, ligand density can be determined sooner.

In embodiments where the five-channel microfluidic system (200) in FIG. 2 is used, immobilizing the ligand on the sensor surfaces of the plurality of sensing channels with different ligand densities can include delivering the ligand through the inlet (230) with the valve port (225) open. Because the valve port (225) is open, the sensing channel (215), the sensing channel farthest away from the inlet (230), can be unexposed to the ligand and can serve as a reference channel for subtracting background noise from the remaining sensing channels (211, 212, 213, and 214). For example, the method can include subtracting the background noise measured at the reference channel (215) from each response signal measured in each of the sensing channels (211, 212, 213, and 214). The method further can include opening another one of the remaining valve ports that are proximate to the inlet (230), relative to the valve port (e.g., the valve port (225)), one by one in sequence so that the respective time for each of the remaining sensing channels to be exposed to the ligand differs.

For example, after a predetermined time interval (t) passes since the ligand was delivered from the inlet (230) in FIG. 2 to the sensing channels (211, 212, 213, and 214) and the valve port (225) was opened, the valve port (225) can be closed, and the valve port (224) can be opened so that the sensing channel (214) is no longer exposed to the ligand, while the ligand is still delivered to the sensing channels (211, 212, and 213). After a second time interval (t) passes (2t after the ligand is delivered to the sensing channels (211, 212, and 213)), the valve port (224) can be closed, and the valve port (223) can be opened so that the ligand is only delivered to the sensing channels (211 and 212). The steps can repeat so that the ligand is only delivered to the sensing channel (211) after the third time interval (t) until the fourth time interval (t), and then valve port (221) is opened, and no channel is exposed to the ligand. After these steps, the ligand can be immobilized in a graduated manner onto the four sensing channels (211, 212, 213, and 214) with the sensing channel (211) having the highest ligand density. In similar or other embodiments, immobilizing the ligand on the sensor surfaces of the plurality of sensing channels with different ligand densities can include injecting each sensor surface with a different ligand solution of a different ligand density (e.g., 0.500, 0,750, 1.000, and 1.250 ng/mm²).

In some embodiments, injecting the analyte solution of the predetermined concentration to the sensing channels can include injecting the analyte solution from an inlet of the multi-channel microfluidic system (e.g., the inlet (240) of the five-channel microfluidic system (200) in FIG. 2) serially to each sensing channel, in an opposite flow direction from that of the ligand solution(s) in the immobilization step (e.g., injecting from the inlet (230) in FIG. 2).

In a number of embodiments, determining the optimal, or near optimal, ligand density based on the respective binding response signal on each sensing channel can include measuring the respective binding response signal on each sensing channel and determining that the optimal, or near optimal, ligand density is the respective ligand density of the sensing channel with the greatest binding response signal among the sensing channels. This optimal, or near optimal, ligand density can vary based on the sizes of the ligand and/or analyte as well as their binding affinity. Generally, the greater a ligand density is, the greater the binding response signal would be because there are more ligands to bind with more analyte species per sensing area. However, such a relationship may not exist when the ligand density reaches a certain point because the crowded ligands begin to impose steric hindrance to the binding reaction. See the sensorgrams for the response signals (310, 320, and 340) in FIG. 3 when their respective ligand densities are 0.500 ng/mm², 0.750 ng/mm², and 1.000 ng/mm². But when the respective ligand density is 1.250 ng/mm², the respective binding response signal (e.g., the response signal (330)) is lower than the respective binding response signal for 1.000 ng/mm² (e.g., the response signal (340)). In this example, the optimal, or near optimal, ligand density is therefore the one that yields the highest binding response signal (e.g., the response signal (340) in FIG. 3).

In several embodiments, determining the optimal or near optimal reaction time (tₒₚₜ) for measuring analyte concentrations (e.g., block 820 in FIG. 8) further can include: (a) injecting analyte standards of varying concentrations (C) to the sensing channels with pre-immobilized ligand with a predetermined ligand density (e.g., 1.000 ng/mm², the optimal or near optimal ligand density determined above, etc.); (b) plotting a respective sensorgram of response signal (R) versus time (t) for each sensing channel (e.g., sensorgrams (410) in FIG. 4); (c) plotting a candidate calibration curve of response signal (R) versus analyte concentration (C) for each candidate optimal reaction time (e.g., candidate calibration curves (510, 520, and/or 530) in FIG. 5) based on the sensorgrams for the sensing channels; and (d) determining the optimal or near optimal reaction time (tₒₚₜ) as a candidate optimal reaction time among the candidate optimal reaction times based on a respective coefficient of determination (r²) and a respective intercept of a respective linear regression for each candidate calibration curve.

In the example shown in FIGS. 4 and 5, three or more candidate optimal reaction times (e.g., lines (420, 430, and/or 440) in FIG. 4) are chosen, and each of the candidate optimal reaction times can be less than 60 seconds and/or within 10 seconds or less apart from each other. The response values at the intersecting points with lines (420), (430), and/or (440) in FIG. 4 respectively are used to plot the candidate calibration curves (510), (520), and/or (530) in FIG. 5 of response values (R) versus analyte concentration (C) at each candidate optimal reaction time in FIG. 5. The calibration curve can be chosen from the candidate calibration curves (510), (520), and/or (530) in FIG. 5 based on a respective coefficient of determination (r²) and a respective intercept of a respective linear regression for each candidate calibration curve. For example, the optimal or near optimal reaction time (tₒₚₜ) can be the one for which: (i) a respective coefficient of determination (r²) of a respective linear regression for the candidate calibration curve is closest to one (1.00); and (ii) a respective intercept of the respective linear regression is closest to zero (0) (e.g., the candidate calibration curve (520) in FIG. 5).

Further, when the calibration curve is determined (e.g., the candidate calibration curve (520) in FIG. 5), based on Equation (3) above, the analyte concentration (C) for an unknown sample can be determined by C=R/α, where α=Rₘₐₓkₐt, and t can be identical or similar to the optimal or near optimal reaction time (tₒₚₜ).

In a number of embodiments, determining the capacity or threshold for multiple sample assays for the sensing channel (e.g., block 830 in FIG. 8) can include: (a) injecting an analyte standard of a high analyte concentration (e.g., 5 μg/mL, 20 μg/mL, 95 μg/mL, or greater than 1 μM or 10 μM, etc.) on the sensing channel pre-immobilized with ligand; (b) plotting a sensorgram (e.g., a sensorgram (610) in FIG. 6) for the response signal measured in an association phase at the sensing channel; (c) fitting, via linear regression with a default coefficient of determination (r²), the linear portion of the sensorgram (e.g., a linear portion (620) of sensorgram (610) in FIG. 6) before the sensorgram curves away; and (d) determining the threshold (e.g., R_{threshold} (630) in FIG. 6) for the sensing channel as the response signal at the point on the sensorgram that deviates from the linear regression line (e.g., the threshold (630) in FIG. 6). The coefficient of determination (r²) can be any suitable value (e.g., 0.90, 0.95, or 0.99, etc.), depending on the expected or requested accuracy for the sample assays.

In many embodiments, once the predetermined ligand density (e.g., the optimal or near optimal ligand density determined above), the predetermined reaction time (e.g., the optimal or near optimal reaction time determined above, tₒₚₜ (450) in FIG. 4, or tₒₚₜ (730) in FIG. 7, etc.), and the predetermined threshold (e.g., the threshold determined above, R_{threshold} (630) in FIG. 6, or R_{threshold} (710) in FIG. 7, etc.) are known or determined by one or more of the acts above, the method can be used to analyze real samples. As stated above, the method can include: (a) immobilizing all of the sensing channels with a ligand of the predetermined ligand density (e.g., block 840 in FIG. 8); (b) measuring increments in a respective response signal in each of the sensing channels based on the predetermined reaction time for each sample injection and the predetermined threshold for a cumulative binding signal (e.g., block 850 in FIG. 8); and (c) determining an analyte concentration of the sample injections based on the measured increments (e.g., block 860 in FIG. 8).

Immobilizing all of the multiple sensing channels (e.g., the sensing channel(s) (210) in FIG. 2)) with the ligand of the predetermined ligand density (e.g., block 840 in FIG. 8) can include injecting a ligand solution of the predetermined ligand density on the multiple sensing channels (e.g., the sensing channels (210, 211, 212, 213, 214, and/or 215) of the system (200) in FIG. 2).

Measuring the increments (e.g., ΔR₁, ΔR₂, ΔR₃, ΔR₄, and/or ΔR₅ (720) in FIG. 7) in the respective response signal in each of the sensing channels (e.g., block 850 in FIG. 8) can include the acts of: (a) injecting a sample to a first sensing channel (e.g., the sensing channel (211) of the system (200) in FIG. 2) for a binding interaction between the ligand immobilized on the first sensing channel and an analyte in the sample for the predetermined reaction time (e.g., tₒₚₜ(450) in FIG. 4 or tₒₚₜ(730) in FIG. 7); (b) measuring a response signal increment at the response signal plateaus (e.g., ΔR₁ (720) in FIG. 7); (c) repeating the acts (a) and (b) until the cumulative response signal for the first sensing channel (e.g., a sum of ΔR₁, ΔR₂, ΔR₃, ΔR₄, and ΔR₅ (720) in FIG. 7) reaches the predetermined threshold (e.g., R_{threshold} (630) in FIG. 6, R_{threshold} (710) in FIG. 7, etc.); and (d) repeating acts (a), (b), and (c) for the next one of the following available sensing channels (e.g., the sensing channels (212, 213, 214, and 215) of the system (200) in FIG. 2) until all of the sensing channels have reached the predetermined threshold.

In embodiments where the system (200) in FIG. 2 is used, the act of injecting the sample to the first sensing channel (e.g., the sensing channel (211)) can include opening a valve port (e.g., the valve port (222)) between the first sensing channel (e.g., the sensing channel (211)) and the second sensing channel (e.g., the sensing channel (212)) and then injecting the sample from an inlet (e.g., the inlet (230)). After the cumulative response signal in the first sensing channel (e.g., the sensing channel (211)) reaches the predetermined threshold, the method can include closing the valve port (e.g., the valve port (222)) between the first sensing channel (e.g., the sensing channel (211)) and the second sensing channel (e.g., the sensing channel (212)), and injecting the sample to the second sensing channel (e.g., the sensing channel (212)) by opening the valve port (e.g., the valve port (223)) between second and third sensing channels (e.g., the sensing channels (212 and 213)). Then the same process can continue until each of the sensing channels has reached the predetermined threshold, as stated above.

In a number of embodiments, determining the analyte concentration of the sample injections (e.g., block 860 in FIG. 8) can include determining the analyte concentration (C) based on the increments in the response signal (ΔR) and a calibration coefficient (a) in C=ΔR/α (Equation 3). As stated above, the calibration coefficient (a) equals Rₘₐₓkₐt, and t is the predetermined reaction time (e.g., the optimal or near optimal reaction time (tₒₚₜ)).

Various embodiments can include a method for measuring a concentration of an analyte through an interaction between the analyte with a ligand. The method can comprise: (a) modifying a sensor surface by immobilizing the ligand on the sensor surface; (b) delivering the analyte via a microfluidic system (e.g., the five-channel microfluidic system (200) in FIG. 2) onto the sensor surface; (c) measuring, via an electronic device (e.g., a scale or a SPR device), a property change (e.g., a mass change, a SPR signal change, or a physical property change) of the sensor surface; and (d) recording the property change with a computing device (e.g., a portable electronic device, a personal computer, a cloud-based server, etc.) for data analysis.

The method further can include determining an optimal or near optimal ligand density on the senor surface by: (a) using a graduated ligand immobilization procedure, as stated above, to immobilize the ligand with different ligand densities on one or more sensing channels of the sensor surface; and (b) selecting the density of the different ligand densities that yields the highest binding response between the ligand and the analyte with a predetermined concentration (typical ranging from 0.01-1 μg/mL for antibody molecules).

The method also can include determining an optimal or near optimal reaction time (e.g., tₒₚₜ(450) in FIG. 4 or tₒₚₜ(730) in FIG. 7) based on a calibration curve determined by: (a) delivering a series of analyte standards of varying analyte concentrations (C) to the sensing channel(s); (b) determining the corresponding sensorgrams based on the response signals measured on the sensing channel(s); (c) overlaying all sensorgrams; (d) selecting three or more small time increments (tᵢ) (each typically being 1-10 seconds apart from each other) to obtain the intersecting response value for each analyte concentration (C); (e) plotting diagrams of the response values for each t, versus C; (f) performing linear regressions to all of the diagrams; and (g) choosing the diagram whose corresponding linear regression plot includes an r² value being closest to 1.00 and an intercept being closest to zero as a calibration curve. The slope (a) of the diagram can be the calibration coefficient, and the time t, for the calibration curve can be the optimal or near optimal reaction time tₒₚₜ (typically <60 seconds).

The method additionally can include determining a measurement capacity threshold (e.g., R_{threshold} (630) in FIG. 6) by: (a) delivering a high analyte concentration (e.g., 1 μg/mL or 1 μM) onto the sensor surface immobilized by the ligand with the optimal or near optimal ligand density; (b) determining a sensorgram based on the response signals measured; (c) performing a linear regression, with r² preset at 0.95, to fit the linear portion of the sensorgram at the early stage of the association phase before the sensorgram curves away; and (d) determining the threshold as the response value at the point on the sensorgram that deviates from the linear regression line.

The method further can include: (a) delivering multiple unknown samples quickly into each sensing channel sequentially at the interval of the optimal or near optimal reaction time (tₒₚₜ) to generate a staircase-like sensorgram (see, FIG. 7); (b) determining the analyte concentration for an unknown sample based on Cₙ=ΔRₙ/α, where n is the sequence number of the injection of the unknown sample; and (c) when a cumulative response signal (R), after n sample injections, is greater than the threshold (R_{threshold}) determined above, switching to a new sensing channel and continuing until all sensing channels have reached the threshold (R_{threshold}). At this point, the method further can include replacing the sensor with a new sensor (e.g., a disposable sensor) or performing a regeneration on the sensor surface of the sensor so that sample measurements can continue.

Various embodiments can include a method. The method can include determining one or more characteristics of an analyte. The one or more characteristics of the analyte can include a mass, a concentration, a dielectric constant, a refractive index, and/or a viscoelasticity, etc.

Many embodiments can include a method. The method can include: (a) immobilizing some or all sensing channels of a sensor with a ligand of a ligand density, (b) measuring increments in a respective response signal in some or all of the sensing channels based on a reaction time for one or more sample injections and a threshold for a cumulative binding signal; and (c) determining a respective analyte concentration of the one or more sample injections.

Various embodiments can include a method. The method can include one or more of: (a) determining an optimal or near optimal ligand density for a ligand; (b) determining an optimal or near optimal reaction time for measuring analyte concentrations; or (c) determining a threshold for multiple sample assays for a sensing channel.

Various embodiments can include a method. The method can include: (a) determining an optimal or near optimal ligand density for a ligand; (b) determining an optimal or near optimal reaction time for measuring analyte concentrations; and (c) determining a threshold for multiple sample assays for a sensing channel. The method further can include: (a) immobilizing some or all sensing channels of a sensor with a ligand of the optimal or near optimal ligand density, (b) measuring increments in a respective response signal in some or all of the sensing channels based on the optimal or near optimal reaction time for one or more sample injections and a threshold for a cumulative binding signal; and (c) determining a respective analyte concentration of the one or more sample injections.

In many embodiments, the analyte and ligand can be any molecules, proteins, and cells. The different sensing channels of the sensor can be immobilized with the same ligand of the same ligand density or different ligands of various densities. Moreover, the analyte captured by the immobilized ligand can be tagged with a label or untagged.

Various embodiments of this invention can be advantageous in that no regeneration is needed until all of the sensing channels have reached the threshold (R_{threshold}). Each sensing channel can be used multiple times without regeneration between tests, until all sensing channels have reached the threshold (R_{threshold}). This process shortens the total analysis time, compared to the conventional method, which requires one or more sensor regenerations for multiple tests. When a specific biomarker or chemical species is to be analyzed in multiple samples, the embodiments further can improve the sample throughput by the method immobilizing the same ligand of the optimal or near optimal density to all sensing channels.

Various embodiments further can include a method. The method can include: (a) measuring increments in a response signal (e.g., block 850 in FIG. 8) in multiple sample injection sessions in a sensing channel until the response signal reaches a threshold response capacity (e.g., R_{threshold} (630) in FIG. 6 or R_{threshold} (710) in FIG. 7); and (b) determining an analyte concentration of the sample (e.g., block 860 in FIG. 8) based at least in part on the increments in the response signal, as measured. In some embodiment, determining the analyte concentration of the sample further can include determining the analyte concentration of the sample further based at least in part on a calibration coefficient associated with the predetermined reaction time. For example, the analyte concentration can be determined based on C=ΔR/α, where the calibration coefficient α =Rₘₐₓkₐt Rₘₐₓkₐtₒₚₜ, as described above.

In many embodiments, measuring the increments in the response signal (e.g., ΔR₁, ΔR₂, ΔR₃, ΔR₄, and/or ΔR₅ (720) in FIG. 7) can include starting a respective sample injection session of the multiple sample injection sessions (e.g., the 5 sample injection sessions for ΔR&, ΔR₂, ΔR₃, ΔR₄, and ΔR₅ (720) in FIG. 7, respectively) by injecting, via a valve port for the sensing channel (e.g., a valve port (220, 221, 222, 223, 224, 225, or 226) in FIG. 2), a sample with an analyte to the sensing channel (e.g., a sensing channel (211, 212, 213, 214, or 215) in FIG. 2). A ligand can be pre-immobilized on a sensor surface (e.g., a sensor surface (110, 120, or 130) in FIG. 1) in the sensing channel. Each of the multiple sample injection sessions can be associated with a predetermined reaction time for the sample (e.g., a predetermined reaction time, tₒₚₜ (450) in FIG. 4 or tₒₚₜ (730) in FIG. 7).

In some embodiments, measuring the increments in the response signal further can include after injecting the sample for the predetermined reaction time, controlling the valve port to terminate the respective sample injection session. Measuring the increments in the response signal additionally can include after terminating the respective sample injection session, measuring, via a response sensing system for the sensor surface, the response signal based on a reaction between the sample and the ligand. Furthermore, measuring the increments in the response signal can include upon determining that the response signal, as measured, is not greater than the threshold response capacity: determining a respective response increment of the increments for the respective sample injection session, and starting a subsequent session of the multiple sample injection sessions for determining a subsequent increment of the increments.

In many embodiments, the method further can include before measuring the increments in the response signal: (a) determining a selected ligand density for the ligand among multiple candidate ligand densities (e.g., block 810 in FIG. 8); (b) injecting a ligand solution of the selected ligand density for the ligand, as determined, to the sensing channel (e.g., block 840 in FIG. 8); and (c) immobilizing the ligand on the sensor surface. Determining the selected ligand density for the ligand among the multiple candidate ligand densities can include: (a) delivering, via a multi-channel microfluidic system (e.g., a five-channel microfluidic system (200) in FIG. 2) with multiple sensing channels, a respective ligand solution for the ligand with a respective density of the multiple candidate ligand densities onto each of multiple sensor surfaces for the multiple sensing channels, wherein the multiple sensing channels comprise the sensing channel, and wherein the multiple sensor surfaces comprise the sensor surface; (b) injecting, via the multi-channel microfluidic system, an analyte solution of a predetermined concentration of the analyte to the multiple sensor surfaces; (c) measuring, via the response sensing system for the multiple sensor surfaces, a respective binding response signal on each of the multiple sensor surfaces; and (d) determining the selected ligand density based on the respective binding response signal on each of the multiple sensing channels.

In a number of embodiments, the method further can include before measuring the increments in the response signal, determining the predetermined reaction time (e.g., block 820 in FIG. 8) based at least in part on a calibration curve of response signal versus analyte concentration for the predetermined reaction time (e.g., a calibration curve (510, 520, or 530) in FIG. 5). Determining the predetermined reaction time can include: (a) injecting, via the multi-channel microfluidic system (e.g., the 5-channel microfluidic system (200) in FIG. 2), analyte standards of multiple known analyte concentrations onto the multiple sensor surfaces, on which the ligand can be pre-immobilized, for the multiple sensing channels of the multi-channel microfluidic system; (b) measuring, via the response sensing system for the multiple sensor surfaces, a respective binding response signal on each of the multiple sensor surfaces; (c) plotting a respective sensorgram of response signal versus time for each of the multiple sensor surfaces (e.g., sensorgrams (410) in FIG. 4); (d) plotting a respective calibration curve of response signal versus analyte concentration for each of multiple candidate reaction times based on the respective sensorgram for each of the multiple sensor surfaces; and (e) determining the predetermined reaction time among the multiple candidate reaction times based on a respective coefficient of determination (r²) and a respective intercept of a respective linear regression for the respective calibration curve for each of the multiple candidate reaction times.

In several embodiments, the predetermined reaction time, as determined, can be associated with: (a) the respective coefficient of determination of the respective linear regression for the respective calibration curve closest to one, relative to other candidate reaction times of the multiple candidate reaction times, and (b) the respective intercept of the respective linear regression is closest to zero, relative to the other candidate reaction times of the multiple candidate reaction times.

In many embodiments, the method further can include before measuring the increments in the response signal, determining the threshold response capacity (e.g., block 830 in FIG. 8) based at least in part on a sensorgram of response signal versus time for a binding interaction between the analyte and the ligand (e.g., a sensorgram (610) in FIG. 6). Determining the threshold response capacity can include: (a) delivering the analyte of a known analyte concentration onto the sensor surface immobilized with the ligand; (b) plotting a sensorgram of response signal versus time based on the response signal measured on the sensor surface; (c) performing a linear regression to fit a linear portion (e.g., a linear portion (620) in FIG. 6) of the sensorgram at an association phase before the sensorgram curves away from the linear portion; and (d) determining the threshold response capacity (e.g., R_{threshold} (630) in FIG. 6) as a response signal reading at or near a point on the sensorgram that deviates from a line for the linear regression.

In many embodiments, measuring the increments in the response signal in the multiple sample injection sessions in the sensing channel further can include measuring, via the response sensing system, respective additional increments in a respective response signal in each of one or more additional sensing channels (e.g., the sensing channels (210, 211, 212, 213, 214, and/or 215) FIG. 2) until the respective response signal reaches the threshold response capacity (e.g., R_{threshold} (630) in FIG. 6 or R_{threshold} (710) in FIG. 7). Determining the analyte concentration of the sample further can include determining the analyte concentration of the sample further based at least in part on the respective additional increments for each of the one or more additional sensing channels, as determined.

Various embodiments also can include a method. The method can include: (a) measuring respective increments in a respective response signal in respective multiple sample injection sessions in each of the sensing channels of a multi-channel microfluidic system (e.g., block 850 in FIG. 8) until the respective response signal reaches a threshold response capacity (e.g., R_{threshold} (630) in FIG. 6 or R_{threshold} (710) in FIG. 7); and (b) determining an analyte concentration of the sample based at least in part on the respective increments, as measured, for each of the sensing channels (e.g., block 860 in FIG. 8). For example, in some embodiments, measuring the respective increments can include measuring the increments in a respective response signal in each of the sensing channels based on (a) a predetermined reaction time for each sample injection and (b) a predetermined threshold for a cumulative binding signal. In the same or different embodiments, determining the analyte concentration can include determining the analyte concentration of each sample injection. Determining the analyte concentration of the sample further can include determining the analyte concentration of the sample further based at least in part on a calibration coefficient (a) associated with the predetermined reaction time (tₒₚₜ). For example, the calibration coefficient (a) can be proportional to the predetermined reaction time (tₒₚₜ).

In many embodiments, measuring the respective increments in the respective response signal in the respective multiple sample injection sessions in each of the sensing channels can include starting a respective sample injection session of the respective multiple sample injection sessions (e.g., a sample injection session for ΔR₁, ΔR₂, ΔR₃, ΔR₄, or ΔR₅ (720) in FIG. 7) for each of the sensing channels (e.g., the sensing channels (210, 211, 212, 213, 214, and/or 215) FIG. 2) by injecting, via the multi-channel microfluidic system (e.g., the 5-channel microfluidic system (200) in FIG. 2), a sample with an analyte to the sensing channels sequentially (e.g., injecting the sample to the sensing channel (211) in FIG. 2 first, then to the sensing channel (212) in FIG. 2, then to the sensing channel (213), etc.). A ligand can be pre-immobilized on a respective sensor surface in each of the sensing channels for the multi-channel microfluidic system. Each of the respective multiple sample injection sessions for each of the sensing channels can be associated with a predetermined reaction time for the sample (e.g., tₒₚₜ (450) in FIG. 4 or tₒₚₜ (730) in FIG. 7).

In a number of embodiments, measuring the respective increments further can include after injecting the sample for the predetermined reaction time, controlling a respective valve port for each of the sensing channels (e.g., the valve ports (220, 221, 222, 223, 224, 225, and/or 226) in FIG. 2) to terminate the respective sample injection session. For example, the sample injection session for the sensing channel (213) in FIG. 2 can be terminated when the sample is injected from the inlet (230) and the valve port (223) is open. In some embodiments, measuring the respective increments further can include after terminating the respective sample injection session, measuring, via a response sensing system (e.g., a SPR device), the respective response signal for each of the sensing channels based on a respective reaction between the sample and the ligand in each of the sensing channels.

In many embodiments, measuring the respective increments further can include upon determining that the respective response signal, as measured, is not greater than the threshold response capacity: (a) determining a respective response increment of the respective increments for the respective sample injection session for each of the sensing channels; and (b) starting a respective subsequent session of the respective multiple sample injection sessions for determining a respective subsequent increment of the respective increments for each of the sensing channels.

In some embodiments, the method further can include before measuring the respective increments in the respective response signal: (a) determining a selected ligand density for the ligand among multiple candidate ligand densities (e.g., block 810 in FIG. 8); (b) injecting a ligand solution of the selected ligand density for the ligand, as determined, to each of the sensing channels sequentially (e.g., block 840 in FIG. 8); and (c) immobilizing the ligand on the respective sensor surface for each of the sensing channels. In some embodiments, the selected ligand density can be one or more optimal or near optimal ligand density or densities, and in the same or different embodiments, immobilizing the ligand can include immobilizing all of the one or more sensing channels with the ligand.

In several embodiments, determining the selected ligand density for the ligand among the multiple candidate ligand densities further can include: (a) delivering, via the multi-channel microfluidic system, a respective ligand solution for the ligand with a respective density of the multiple candidate ligand densities onto the respective sensor surfaces for each of the sensing channels; (b) injecting, via the multi-channel microfluidic system, an analyte solution of a predetermined concentration of the analyte to the respective sensor surface for each of the sensing channels; (c) measuring, via the response sensing system, a respective first response signal on the respective sensor surface for each of the sensing channels; and (d) determining the selected ligand density based on the respective first response signal on the respective sensor surface for each of the sensing channels.

In many embodiments, the method further can include before measuring the respective increments in the respective response signal for each of sensing channels, determining the predetermined reaction time based at least in part on a calibration curve of response signal versus analyte concentration for the predetermined reaction time (e.g., block 820 in FIG. 8). In some embodiments, determining the predetermined reaction time can include determining one or more optimal or near optimal reaction time(s) for measuring one or more analyte concentrations. Determining the predetermined reaction time further can include: (a) injecting, via the multi-channel microfluidic system, analyte standards of multiple known analyte concentrations onto the respective sensor surface for each of the sensing channels, wherein the ligand can be pre-immobilized on the respective sensor surface for each of the sensing channels; (b) measuring, via the response sensing system, a respective binding response signal on the respective sensor surface for each of the sensing channels; (c) plotting a respective sensorgram of response signal versus time for each of the sensing channels; (d) plotting a respective calibration curve of response signal versus analyte concentration for each of multiple candidate reaction times based on the respective sensorgram for each of the sensing channels; and (e) determining the predetermined reaction time among the multiple candidate reaction times based on a respective coefficient of determination and a respective intercept of a respective linear regression for the respective calibration curve for each of the multiple candidate reaction times.

In some embodiments, the predetermined reaction time, as determined, can be associated with: (a) the respective coefficient of determination (r²) of the respective linear regression for the respective calibration curve closest to one, relative to other candidate reaction times of the multiple candidate reaction times (e.g., r²=0.999 for the calibration curve (520) in FIG. 5), and (b) the respective intercept of the respective linear regression is closest to zero, relative to the other candidate reaction times of the multiple candidate reaction times (e.g., the calibration curves (510 and 520) in FIG. 5).

In many embodiments, the method further can include before measuring the respective increments in the respective response signal for each of sensing channels, determining the threshold response capacity (e.g., block 830 in FIG. 8) based at least in part on a sensorgram of response signal versus time for a binding interaction between the analyte and the ligand. In some embodiments, determining the threshold response capacity can include determining one or more capacities or thresholds for multiple sample assays for one or more sensing channels. Determining the threshold response capacity further can include: (a) delivering the analyte of a known analyte concentration onto the respective sensor surface for each of the sensing channels immobilized with the ligand; (b) plotting a respective sensorgram of response signal versus time based on the respective response signal measured on the respective sensor surface; (c) performing a linear regression to fit a respective linear portion of the respective sensorgram at a respective association phase before the respective sensorgram curves away from the respective linear portion; and (d) determining the threshold response capacity as a respective response reading at or near a respective point on the respective sensorgram that deviates from a respective line for the linear regression.

Various embodiments additionally can include a method. The method can include: (a) determining a session reaction time for a sample with an analyte based at least in part on a calibration curve of surface plasmon resonance (SPR) signal versus analyte concentration for the session reaction time (e.g., block 820 in FIG. 8); (b) measuring increments in an SPR response signal in multiple sample injection sessions in a sensing channel until the SPR response signal reaches a threshold response capacity (e.g., block 850 in FIG. 8); and (c) determining an analyte concentration of the sample based at least in part on the increments in the SPR response signal, as measured (e.g., block 860 in FIG. 8).

In many embodiments, measuring the increments in the SPR response signal further can include starting a respective sample injection session of the multiple sample injection sessions by injecting the sample to the sensing channel, wherein: a ligand can be pre-immobilized on a sensor surface in the sensing channel; and each of the multiple sample injection sessions can be associated with the session reaction time for the sample. Additionally, measuring the increments in the SPR response signal can include after injecting the sample for the session reaction time, controlling a valve port to terminate the respective sample injection session. Measuring the increments in the SPR response signal also can include after terminating the respective sample injection session, measuring, via an SPR sensing system for the sensor surface, the SPR response signal based on a reaction between the sample and the ligand. Moreover, measuring the increments in the SPR response signal can include upon determining that the SPR response signal, as measured, is not greater than the threshold response capacity: (a) determining an SPR respective response increment of the increments for the respective sample injection session; and (b) starting a subsequent session of the multiple sample injection sessions for determining a subsequent increment of the increments.

The embodiments of this invention further can be advantageous when multiple analytes in a complex sample medium are to be analyzed. Each of the different sensing channels can be immobilized with a respective ligand that specifically captures a respective analyte. The samples to be analyzed can be introduced with all sensing channels open, and the analytes can be simultaneously measured in each injection.

Moreover, the embodiments of the present invention can be advantageous because the method(s) and/or the acts can be entirely, or at least in part, implemented via execution of computing instructions configured to run at one or more processors and stored at one or more non-transitory computer-readable media. The method further can provide an analytical method for chemical and biological assays. In embodiments using a pre-programed auto-sampler and/or computer-controlled channel opening and closing, the computer-implemented method(s) can automatically and continuously analyze assays of hundreds of samples in an unattended manner for days and/or weeks. For example, in embodiments where the method is implemented by a computer and uses four analysis channels sequentially over a single tris-NTA sensor immobilized with a Si protein, at least 20 cycles of surface generations can be achieved on a single sensor for assaying at least 800 serum samples (the exact number of assays is dependent on the antibody concentrations in sera).

In many embodiments, the techniques described herein can provide a practical application and several technological improvements. The techniques described herein can provide improved approaches for determining analyte concentration(s) in multiple samples. Specifically, the techniques disclosed here can determine the analyte concentration(s) more quickly compared to conventional approaches. The analysis can be based on response signals measured at an early state of an association phase, and the time used for gathering data thus can be reduced. Further, the analyte concentration(s) is/are generally linearly proportional to the response signal value at the early stage of the association phase, and calculating the analyte concentration(s) can be faster because the equation can be simplified. Moreover, by using increments (e.g., not only the first early stage, but multiple early stages) in the response signal in each sensing channel to determine the analyte concentration(s), the approaches described here can reduce sample consumption while increasing data gathered.

Although measuring binding interactions has been described with reference to specific embodiments, it will be understood by those skilled in the art that various changes may be made without departing from the spirit or scope of the disclosure. For example, the methods and/or algorithms described in the embodiments above can be applied to assays of different analytes in a mixture sample. In such an application, different ligands can be separately immobilized to different sensing channels with their respective optimal or near optimal surface densities. The thresholds for response signals and calibration curves can then be determined individually. When unknown samples are delivered, all sensing channels can be open so that concentrations of the respective analytes can be simultaneously determined.

Replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

What is claimed is:

1. A method comprising:
measuring increments in a response signal in multiple sample injection sessions in a sensing channel until the response signal reaches a threshold response capacity, comprising:
starting a respective sample injection session of the multiple sample injection sessions by injecting, via a valve port for the sensing channel, a sample with an analyte to the sensing channel, wherein:
a ligand is pre-immobilized on a sensor surface in the sensing channel; and
each of the multiple sample injection sessions is associated with a predetermined reaction time for the sample;
after injecting the sample to the sensing channel for the predetermined reaction time, controlling the valve port to terminate the respective sample injection session;
after terminating the respective sample injection session, measuring, via a response sensing system for the sensor surface, the response signal based on a reaction between the sample and the ligand; and
upon determining that the response signal, as measured, is not greater than the threshold response capacity:
determining a respective response increment of the increments for the respective sample injection session; and
starting a subsequent session of the multiple sample injection sessions for determining a subsequent increment of the increments; and
determining an analyte concentration of the sample based at least in part on the increments in the response signal, as measured.

2. The method in claim 1 further comprising before measuring the increments in the response signal:
determining a selected ligand density for the ligand among multiple candidate ligand densities;
injecting a ligand solution of the selected ligand density for the ligand, as determined, to the sensing channel; and
immobilizing the ligand on the sensor surface in the sensing channel.

3. The method in claim 2, wherein determining the selected ligand density for the ligand among the multiple candidate ligand densities further comprises:
delivering, via a multi-channel microfluidic system with multiple sensing channels, a respective ligand solution for the ligand with a respective density of the multiple candidate ligand densities onto each of multiple sensor surfaces for the multiple sensing channels, wherein the multiple sensing channels comprise the sensing channel, and wherein the multiple sensor surfaces comprise the sensor surface;
injecting, via the multi-channel microfluidic system, an analyte solution of a predetermined concentration of the analyte to the multiple sensor surfaces;
measuring, via the response sensing system for the multiple sensor surfaces, a respective binding response signal on each of the multiple sensor surfaces; and
determining the selected ligand density based on the respective binding response signal on each of the multiple sensing channels.

4. The method in claim 3 further comprising:
before measuring the increments in the response signal, determining the predetermined reaction time based at least in part on a calibration curve of response signal versus analyte concentration for the predetermined reaction time.

5. The method in claim 4, wherein determining the predetermined reaction time further comprises:
injecting, via the multi-channel microfluidic system, analyte standards of multiple known analyte concentrations onto the multiple sensor surfaces for the multiple sensing channels of the multi-channel microfluidic system, wherein:
the ligand is pre-immobilized on each of the multiple sensor surfaces;
measuring, via the response sensing system for the multiple sensor surfaces, a respective binding response signal on each of the multiple sensor surfaces;
plotting a respective sensorgram of response signal versus time for each of the multiple sensor surfaces;
plotting a respective calibration curve of response signal versus analyte concentration for each of multiple candidate reaction times based on the respective sensorgram for each of the multiple sensor surfaces; and
determining the predetermined reaction time among the multiple candidate reaction times based on a respective coefficient of determination and a respective intercept of a respective linear regression for the respective calibration curve for each of the multiple candidate reaction times.

6. The method in claim 5, wherein:
the predetermined reaction time, as determined, is associated with: (a) the respective coefficient of determination of the respective linear regression for the respective calibration curve closest to one, relative to other candidate reaction times of the multiple candidate reaction times, and (b) the respective intercept of the respective linear regression is closest to zero, relative to the other candidate reaction times of the multiple candidate reaction times.

7. The method in claim 1 further comprising:
before measuring the increments in the response signal, determining the threshold response capacity based at least in part on a sensorgram of response signal versus time for a binding interaction between the analyte and the ligand.

8. The method in claim 7, wherein determining the threshold response capacity further comprises:
delivering the analyte of a known analyte concentration onto the sensor surface immobilized with the ligand;
plotting a sensorgram of response signal versus time based on the response signal measured on the sensor surface;
performing a linear regression to fit a linear portion of the sensorgram at an association phase before the sensorgram curves away from the linear portion; and
determining the threshold response capacity as a response signal reading at or near a point on the sensorgram that deviates from a line for the linear regression.

9. The method in claim 1, wherein determining the analyte concentration of the sample further comprises determining the analyte concentration of the sample further based at least in part on a calibration coefficient associated with the predetermined reaction time.

10. The method in claim 1, wherein:
measuring the increments in the response signal in the multiple sample injection sessions in the sensing channel further comprises measuring, via the response sensing system, respective additional increments in a respective response signal in each of one or more additional sensing channels until the respective response signal reaches the threshold response capacity; and
determining the analyte concentration of the sample further comprises determining the analyte concentration of the sample further based at least in part on the respective additional increments for each of the one or more additional sensing channels, as determined.

11. A method comprising:
measuring respective increments in a respective response signal in respective multiple sample injection sessions in each of sensing channels of a multi-channel microfluidic system until the respective response signal reaches a threshold response capacity, comprising:
starting a respective sample injection session of the respective multiple sample injection sessions for each of the sensing channels by injecting, via the multi-channel microfluidic system, a sample with an analyte to the sensing channels sequentially, wherein:
a ligand is pre-immobilized on a respective sensor surface in each of the sensing channels for the multi-channel microfluidic system; and
each of the respective multiple sample injection sessions for each of the sensing channels is associated with a predetermined reaction time for the sample;
after injecting the sample to the sensing channels for the predetermined reaction time, controlling a respective valve port for each of the sensing channels to terminate the respective sample injection session;
after terminating the respective sample injection session, measuring, via a response sensing system, the respective response signal for each of the sensing channels based on a respective reaction between the sample and the ligand in each of the sensing channels; and
upon determining that the respective response signal, as measured, is not greater than the threshold response capacity:
determining a respective response increment of the respective increments for the respective sample injection session for each of the sensing channels; and
starting a respective subsequent session of the respective multiple sample injection sessions for determining a respective subsequent increment of the respective increments for each of the sensing channels; and
determining an analyte concentration of the sample based at least in part on the respective increments, as measured, for each of the sensing channels.

12. The method in claim 11 further comprising, before measuring the respective increments in the respective response signal:
determining a selected ligand density for the ligand among multiple candidate ligand densities;
injecting a ligand solution of the selected ligand density for the ligand, as determined, to each of the sensing channels sequentially; and
immobilizing the ligand on the respective sensor surface for each of the sensing channels.

13. The method in claim 12, wherein determining the selected ligand density for the ligand among the multiple candidate ligand densities further comprises:
delivering, via the multi-channel microfluidic system, a respective ligand solution for the ligand with a respective density of the multiple candidate ligand densities onto the respective sensor surface for each of the sensing channels;
injecting, via the multi-channel microfluidic system, an analyte solution of a predetermined concentration of the analyte to the respective sensor surface for each of the sensing channels;
measuring, via the response sensing system, a respective first response signal on the respective sensor surface for each of the sensing channels; and
determining the selected ligand density based on the respective first response signal on the respective sensor surface for each of the sensing channels.

14. The method in claim 13 further comprising:
before measuring the respective increments in the respective response signal for each of the sensing channels, determining the predetermined reaction time based at least in part on a calibration curve of response signal versus analyte concentration for the predetermined reaction time.

15. The method in claim 14, wherein determining the predetermined reaction time further comprises:
injecting, via the multi-channel microfluidic system, analyte standards of multiple known analyte concentrations onto the respective sensor surface for each of the sensing channels, wherein:
the ligand is pre-immobilized on the respective sensor surface for each of the sensing channels;
measuring, via the response sensing system, a respective binding response signal on the respective sensor surface for each of the sensing channels;
plotting a respective sensorgram of response signal versus time for each of the sensing channels;
plotting a respective calibration curve of response signal versus analyte concentration for each of multiple candidate reaction times based on the respective sensorgram for each of the sensing channels; and
determining the predetermined reaction time among the multiple candidate reaction times based on a respective coefficient of determination and a respective intercept of a respective linear regression for the respective calibration curve for each of the multiple candidate reaction times.

16. The method in claim 15, wherein:
the predetermined reaction time, as determined, is associated with: (a) the respective coefficient of determination of the respective linear regression for the respective calibration curve closest to one, relative to other candidate reaction times of the multiple candidate reaction times, and (b) the respective intercept of the respective linear regression is closest to zero, relative to the other candidate reaction times of the multiple candidate reaction times.

17. The method in claim 11 further comprising:
before measuring the respective increments in the respective response signal for each of the sensing channels, determining the threshold response capacity based at least in part on a sensorgram of response signal versus time for a binding interaction between the analyte and the ligand.

18. The method in claim 17, wherein determining the threshold response capacity further comprises:
delivering the analyte of a known analyte concentration onto the respective sensor surface for each of the sensing channels immobilized with the ligand;
plotting a respective sensorgram of response signal versus time based on the respective response signal measured on the respective sensor surface;
performing a linear regression to fit a respective linear portion of the respective sensorgram at a respective association phase before the respective sensorgram curves away from the respective linear portion; and
determining the threshold response capacity as a respective response reading at or near a respective point on the respective sensorgram that deviates from a respective line for the linear regression.

19. The method in claim 11, wherein determining the analyte concentration of the sample further comprises determining the analyte concentration of the sample further based at least in part on a calibration coefficient associated with the predetermined reaction time.

20. A method comprising:
determining a session reaction time for a sample with an analyte based at least in part on a calibration curve of surface plasmon resonance (SPR) signal versus analyte concentration for the session reaction time;
measuring increments in an SPR response signal in multiple sample injection sessions in a sensing channel until the SPR response signal reaches a threshold response capacity, comprising:
starting a respective sample injection session of the multiple sample injection sessions by injecting the sample to the sensing channel, wherein:
a ligand is pre-immobilized on a sensor surface in the sensing channel; and
each of the multiple sample injection sessions is associated with the session reaction time for the sample;
after injecting the sample to the sensing channel for the session reaction time, controlling a valve port to terminate the respective sample injection session;
after terminating the respective sample injection session, measuring, via an SPR sensing system for the sensor surface, the SPR response signal based on a reaction between the sample and the ligand; and
upon determining that the SPR response signal, as measured, is not greater than the threshold response capacity:
determining an SPR respective response increment of the increments for the respective sample injection session; and
starting a subsequent session of the multiple sample injection sessions for determining a subsequent increment of the increments; and
determining an analyte concentration of the sample based at least in part on the increments in the SPR response signal, as measured.
